(19) United States
(12) Patent Application Publication
(10) Pub. No.: US 2005/0163855 A1
(43) Pub. Date: Jul. 28, 2005

(54) Method of preparing low-crystallinity oltipraz or amorphous oltipraz

(75) Inventors: Cheong Weon Cho, Seongnam-city (KR); Kyoung Rae Kang, Seoul (KR); Sang Ho Lee, Anyang-city (KR); Jeong Ku, Yongin-city (KR); Taekrho Kim, Seoul (KR); Sung Hak Lee, Yongin-city (KR); Jae Mook Choi, Gwangju-city (KR); Jun Hee Cheon, Suwon-city (KR); Tae Kun An, Gochang-gun (KR); Hyun Jung Park, Yongin-city (KR); Eun Kyung Jeon, Yongin-city (KR); Kwang Do Choi, Anyang-city (KR); Jee Woong Lim, Seongnam-city (KR); Kwang Hee Hong, Seoul (KR); Hyesuk Hong, Seongnam-city (KR); Il Hwan Kim, Daejeon-city (KR); Tae Hyoung Kim, Changwon-city (KR); Nak Hyun Choi, Icheon-city (KR); Young Hoon Kim, Seoul (KR); Kyu Jeong Yeon, Yongin-city (KR); Hearan Suh, Icheon-city (KR); Hae Tak Jin, Suwon-city (KR); Jinwan Kim, Seoul (KR); In Ki Min, Yongin-city (KR)

Correspondence Address: ROTHWELL, FIGG, ERNST & MANBECK, P.C., 1425 K STREET, N.W., SUITE 800, WASHINGTON, DC, 20005, US

(73) Assignee: CJ CORPORATION, Seoul (KR)

(21) Appl. No.: 11/043,069

(22) Filed: Jan. 27, 2005

(30) Foreign Application Priority Data
Jan. 27, 2004 (KR) 10-2004-0005000

(51) Int. Cl. A61K 31/724; A61K 31/497; A61K 9/14

(52) U.S. Cl. 424/486; 514/58; 514/255.05

(57) ABSTRACT

Provided is a method of preparing low-crystallinity oltipraz or amorphous oltipraz. The method includes: obtaining a mixed solution containing oltipraz and a water-soluble polymer or a water-insoluble polymer in a solvent, the solvent being an organic solvent or purified water; and solid-dispersing the oltipraz in the polymer. In the solid-dispersing, the mixed solution may be spray dried using a spray dryer or granulated using a fluid bed granulator.

### BACKGROUND OF THE INVENTION

This application claims the benefit of Korean Patent Application No. 10-2004-0005000, filed on Jan. 27, 2004, in the Korean Intellectual Property Office, the disclosure of which is incorporated herein in its entirety by reference.

1. Field of the Invention

The present invention relates to a method of preparing low-crystallinity oltipraz or amorphous oltipraz, and more particularly, to a method of preparing low-crystallinity oltipraz or amorphous oltipraz to increase solubility and bioavailability of oltipraz, which has a low solubility.

2. Description of the Related Art

The liver is an organ with consistent enzymatic reactions and energy metabolism and plays a key role in the metabolism of xenobiotics and in the metabolism of endogenous substances. Among the many chronic diseases that lead to death, liver disease such as hepatitis, cirrhosis, and liver cancer, and cardiovascular diseases are the most widespread. Thus, there is a need to develop therapeutic and prophylactic pharmaceutical compositions which can reduce damage of the liver tissue and be ultimately applied to treat the liver.

Various substances, including several synthetic compounds and galenical preparations, show hepatoprotective functions both in vitro and in vivo. Although it has been known that silymarin and betaine have liver protective effects as a result of the action mechanism of cytokine inhibition and an increase in the level of glutathione, a therapeutic effect would be hard to expect because of its low effectiveness. Because no appropriate treating agents against liver disease are currently available, said agents are used for clinical trials. Malotilate and its derivatives, the indication of which is the treatment of liver fibrosis, protect the liver from toxic chemicals and the possible action mechanism includes the induction of phase II conjugating enzymes and the inhibition of cytochrome P450s. However, the compounds non-selectively inhibit cytochrome P450s and show only prophylactic effects.

It is known that several derivatives of dithiolthione, which naturally occurs in cruciferous vegetables and contains sulfur, have liver protecting effects. Among them, oltipraz was used as a treating agent for schistosomiasis in the early 1980s and is represented by the following formula (KR 2000-0010540):

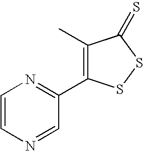

It is reported that oltipraz has therapeutic and prophylactic effects on liver fibrosis and cirrhosis by inhibiting generation of TGF-β (see, Korean Laid-Open Patent Publication Nos. 2001-91012 and 2003-67935). However, oltipraz is lipid-soluble and rarely soluble in water with a water solubility of 1 μg/Ml or less and has high crystallinity. Thus, to increase an effective concentration of oltipraz in blood to a level suitable to exhibit the effects, a relatively large amount of the drug must be orally administered. That is, its dissolution rate in a digestive tract determines an absorption rate in a body.

Conventionally, oltipraz is formulated into a preparation, for example, tablets, powders, capsules, or suspension. Specifically, oltipraz is mixed with a predetermined amount of lactose, starch, or magnesium state, etc. and the mixture is granulated and compressed to form tablets. Alternatively, the resultant granules are filled in a capsule to form a hard capsule.

A method of preparing a soft capsule is described in Korean Laid-Open Patent Publication No. 2003-67935. In this method, a suspension of sucrose, isomerized sugar, flavoring agent, etc. is mixed with polyethylene glycol 400, concentrated glycerin, purified water, etc. to prepare a soft capsule.

Although oltipraz preparations can be prepared in a simple manner using the above methods, the solubility and bioavailability of oltipraz cannot be sufficiently increased.

### SUMMARY OF THE INVENTION

The present invention provides a method of preparing oltipraz having a high solubility and bioavailability.

According to an aspect of the present invention, there is provided a method of preparing low-crystallinity oltipraz or amorphous oltipraz, comprising: obtaining a mixed solution containing oltipraz and a water-soluble polymer or a water-insoluble polymer in a solvent, the solvent being an organic solvent or purified water; and solid-dispersing the oltipraz in the polymer.

According to another aspect of the present invention, there is provided a method of using the low-crystallinity oltipraz or amorphous oltipraz in preparing a tablet or a capsule.

### BRIEF DESCRIPTION OF THE DRAWINGS

The above and other features and advantages of the present invention will become more apparent by describing in detail exemplary embodiments thereof with reference to the attached drawings in which:

FIG. 1 is a graph of time vs. dissolution concentrations for tablets prepared in Comparative Example 1, Example 10, and Example 11;

FIG. 2 is a graph of time vs. concentrations of oltipraz in blood plasma for rats to which a spray dried product prepared in Example 1, a spray dried product prepared in Example 2, and a micronized powder prepared in Comparative Example 1 were respectively orally administered;

FIG. 3A is a scanning electron microscope (SEM) photo of oltipraz powders as a raw material;

FIG. 3B is an SEM photo of oltipraz pulverized using an air jet impact mill;

FIG. 3C is an SEM photo of polyvinylpyrrolidone (molecular weight: 40,000);

FIG. 3D is an SEM photo of a polyvinylpyrrolidone-vinyl acetate copolymer;

FIG. 3E is an SEM photo of a spray dried product prepared in Example 1;

FIG. 3F is an SEM photo of a spray dried product prepared in Example 2;

FIG. 3G is an SEM photo of a spray dried product prepared in Example 5;

FIG. 3H is an SEM photo of a spray dried product prepared in Example 3;

FIG. 3I is an SEM photo of granules prepared in Example 6;

FIG. 3J is an SEM photo of granules prepared in Example 9;

FIG. 3K is an SEM photo of a mixture of oltipraz and polyvinylpirrolidone-vinyl acetate copolymer in a ratio of 3:7;

FIG. 4A is a graph illustrating crystallinity of each of oltipraz as a raw material, polyvinylpyrrolidone (molecular weight: 40,000), and microcrystalline cellulose, measured using an X-ray diffractor; and

FIG. 4B is a graph illustrating crystallinity of each of the spray dried product prepared in Example 1, the spray dried product prepared in Example 5, the granules prepared in Example 6, and the granules prepared in Example 7, measured using an X-ray diffractor.

### DETAILED DESCRIPTION OF THE INVENTION

In an embodiment of the present invention, there is provided a method of preparing low-crystallinity oltipraz or amorphous oltipraz, comprising: obtaining a mixed solution containing oltipraz and a water-soluble polymer or a water-insoluble polymer in a solvent, the solvent being an organic solvent or purified water; and solid-dispersing the oltipraz in the polymer.

In the solid-dispersing, the mixed solution may be spray dried using a spray dryer or granulated using a fluid bed granulator.

The mixed solution may further comprise an absorption enhancer. The absorption enhancer may include at least one compound selected from the group consisting of ascorbic acid, citric acid, xylitol, and polyethylene glycol or its derivative.

The organic solvent used in preparing the mixed solution may be methylene chloride, acetone, chloroform, acetonitrile, methanol, or ethanol, and preferably methylene chloride.

The water-soluble polymer may include at least one polymer selected from the group consisting of polyvinylpyrrolidone or its derivative, a polyvinylpyrrolidone-vinyl acetate copolymer, alginic acid, alginate or its derivative, α-cyclodextrin or its derivative, β-cyclodextrin or its derivative, γ-cyclodextrin or its derivative, polyoxyethylene-polyoxypropylene copolymer, polyethylene glycol or its derivative, polyvinyl alcohol, xanthan gum, arabic gum, or a combination thereof.

The polyvinylpyrrolidone may have a molecular weight of 2,500-3,000,000.

The polyvinylpyrrolidone-vinyl acetate copolymer may have a molecular weight of 30,000-50,000.

The alginate derivative may be an ethylene or propylene derivative of sodium alginate and has a molecular weight of 20,000-200,000.

The β-cyclodextrin derivative may be a propylene derivative of β-cyclodextrin or a metylated derivative of β-cyclodextrin.

The polyoxyethylene-polyoxypropylene copolymer may have an oxyethylene content of 45-75%.

The polyethylene glycol or its derivative may have a molecular weight of 200-90,000.

The polyethylene glycol derivative may be an esterificated derivative of polyethylene glycol.

The water-insoluble polymer may include at least one selected from the group consisting of cellulose or its derivative, polymethacrylate, and polyalkylacrylate.

The cellulose derivative may be cellulose acetate, cellulose acetate phthalate, hydroxypropylene methylcellulose, hydroxypropylene methylcellulose phthalate, ethylcellulose, methylcellulose, or hydroxypropylene cellulose. The cellulose derivative may be hydroxypropylene methylcellulose having a viscosity of 5-50 cps.

In the mixed solution, the concentration of the water-soluble polymer or water-insoluble polymer may be 5-90 parts by weight based on 100 parts by weight of oltipraz. If the concentration of the water-soluble polymer or water-insoluble polymer is less than 5 parts by weight, the low-crystalliny or amorphous oltipraz cannot be obtained. If the concentration of the water-soluble polymer or water-insoluble polymer is greater than 90 parts by weight, a dissolution rate and bioavailability of oltipraz are decreased.

When the mixed solution further comprises the absorption enhancer, the concentration of the water-soluble polymer or water-insoluble polymer in the mixed solution may be 5-90 parts by weight and the concentration of the absorption enhancer in the mixed solution may be 5-90 parts by weight, respectively, based on 100 parts by weight of oltipraz. Preferably, the concentration of the water-soluble polymer or water-insoluble polymer is 45 parts by weight and the concentration of the absorption enhancer is 10 parts by weight, respectively, based on 100 parts by weight of oltipraz. If the concentration of the absorption enhancer is greater than 90 parts by weight, it may take a long time to spray dry the mixed solution due to an increase in a total amount of solvent. Glass transition temperature is measured in the above concentration ranges, when demonstrating that the components are intimately mixed in the above concentration ranges.

The low-crystallinity oltipraz or amorphous oltipraz prepared using the above method may be used in itself or formulated into a tablet or a capsule.

A method of solid-dispersing oltipraz in a polymer according to an embodiment of the present invention will now be described in more detail.

According to a method of preparing low-crystallinity oltipraz or amorphous oltipraz in an embodiment of the present invention, bioavailability of oltipraz, an agent for treating cirrhosis, can be increased. In this method, oltipraz, which is a crystalline and rarely soluble pharmaceutical, a water-soluble polymer or a water-insoluble polymer, and optionally, an absorption enhancer, are dissolved in an organic solvent or purified water, and then, the oltipraz is solid-dispersed in the polymer.

The method of solid-dispersing the oltipraz includes a method in which the mixed solution is spray dried using a spray dryer and a method in which the mixed solution is granulated using a fluid bed granulator. The method of solid-dispersing the oltipraz will now be described in more detail.

The first process: rarely soluble oltipraz is dissolved in an organic solvent or purified water.

The organic solvent may be a volatile solvent, such as methylene chloride, acetone, chloroform, acetonitrile, methanol, or ethanol. The organic solvent is preferably methylene chloride, since oltipraz has a solubility of 7.6 mg/ml in methylene chloride and methylene chloride is less explosive than acetone.

Although oltipraz can be very easily dissolved in an oil-phase liquid polymer, such as polyethylene glycol and polypropylene glycol, and an oily solvent, such as N,N-dimethylformamide and N-methylpyrrolidone, these solvents have a very low volatility, and thus, the spray drying cannot be easily performed. That is, it is advantageous that the organic solvent can easily dissolve oltipraz and is highly volatile.

The solubilities of oltipraz in various organic solvents and oil-phase liquid polymers are listed in Table 1.

TABLE 1
Solubility (mg/ml)

| | | | |
|---|---|---|---|
| Ethanol | 0.67 | Dimethylisosorbide | 17.24 |
| Methanol | 0.50 | N-methypyrrolidone | 13.65 |
| Acetone | 4.05 | Tetraglycol | 13.56 |
| Chloroform | 28.59 | Cremophor RH40 | 10.68 |
| N,N-dimethylformamide | 31.34 | Labrasol | 12.59 |
| Dimethyl sulfoxide | 29.09 | Polymethylene glycol | 10.53 |
| | | 400 | |
| Acetonitrile | 2.73 | Transcutol | 10.34 |
| Methylene chloride | 7.6 | Cremophor EL | 9.44 |
| | | Polymethylene glycol | 7.99 |
| | | 200 | |
| | | Polypropylene glycol | 0.57 |

The second process: a water-soluble polymer or a water-insoluble polymer, alone or together with an absorption enhancer, is dissolved in an organic solvent or purified water.

The water-soluble polymer may include at least one compound selected from the group consisting of polyvinylpyrrolidone or its derivative, polyvinylpyrrolidone-vinyl acetate copolymer, alginic acid, alginate or its derivative, α-cyclodextrin or its derivative, β-cyclodextrin or its derivative, γ-cyclodextrin or its derivative, polyoxyethylene-polyoxypropylene copolymer, polyethylene glycol or its derivative, polyvinyl alcohol, xanthan gum, and arabic gum. Preferably, the water-soluble polymer is polyvinylpyrrolidone having a molecular weight of 40,000-50,000 and a polyvinylpyrrolidone-vinyl acetate copolymer having a molecular weight of 30,000-50,000.

The water-insoluble polymer may include, but are not limited to, at least one compound selected from the group consisting of cellulose or its derivative, polymethacrylate, and polyalkylacrylate.

The cellulose derivative may include, but are not limited to, cellulose acetate, cellulose acetate phthalate, hydroxypropylene methylcellulose, hydroxypropylene methylcellulose phthalate, ethylcellulose, methylcellulose, or hydroxypropylene cellulose. The cellulose derivative may be hydroxypropylene methylcellulose having a viscosity of 5-50 cps. If the viscosity of the cellulose derivative is greater than 50 cps, the spray drying cannot be easily performed, and thus, the desired spray dried product cannot be obtained.

Polymethacrylate and polyalkylacrylate may be used as a combination of at least two components, for example, a combination of polymethacrylate and polymethylmethacrylate in a ratio of 1:1, or a combination of polyethylacrylate, polymethylmethacrylate, and polytrimethylammonioethyl methacrylate chloride in a ratio of 1:2:0.1 or 1:2:0.2.

The solvent which can dissolve the water-soluble polymer or water-insoluble polymer may include at least one solvent selected from the group consisting of ethanol, methanol, methylene chloride, acetonitrile, acetone, isopropyl alcohol, and chloroform. As the purified water, non-ionized purified water is used.

When the water-soluble polymer or water-insoluble polymer is dissolved in the organic solvent, the absorption enhancer may be further added to the organic solvent. The absorption enhancer has a high tendency to form a complex with the main pharmaceutical via a hydrogen bond, and thus allows for the formation of an amorphous state of the pharmaceutical and increases absorption of the pharmaceutical in digestive organ.

The absorption enhancer may be an organic acid. The organic acid may include at least one compound selected from the group consisting of ascorbic acid, citric acid, xylitol, and polyethylene glycol. Preferably, the organic acid is citric acid.

The oltipraz solution and the polymer solution are separately produced in the first process and the second process, respectively. Alternatively, the oltipraz and the water-soluble polymer or water-insoluble polymer, or the oltipraz, the water-soluble polymer or water-insoluble polymer, and the absorption enhancer may be simultaneously dissolved in a solvent to obtain a mixed solution.

The third process: the oltipraz solution is mixed with the aqueous solution of the polymer to obtain a mixed solution.

When the oltipraz solution is mixed with the aqueous solution of the polymer, their mixing ratio may be such that the concentration of the polymer in the mixed solution is 10-90 parts by weight based on 100 parts by weight of oltipraz. Preferably, the mixing ratio of the oltipraz solution and the aqueous solution of the polymer is 3:7.

When the absorption enhancer is further added, their mixing ratio may be such that the concentration of the polymer in the mixed solution is 5-90 parts by weight and the concentration of the absorption enhancer is 5-90 parts by weight, respectively, based on 100 parts by weight of oltipraz. Preferably, the mixing ratio of oltipraz, the polymer, and the absorption enhancer is 100:45:10.

The fourth process: a spray dried product or granules are obtained using the mixed solution containing the oltipraz and the polymer.

The mixed solution is stirred in a mechanical mixer for 30-60 minutes, and then, may be spray dried using a spray dryer to obtain a spray dried product having fine particles.

First, the well-stirred mixed solution may be spray dried using the spray dryer at an inlet temperature of 60-100° C. and an outlet temperature of 40-80° C. An inflow rate of the mixed solution is 300-1500 Ml/hr and may be selected considering a drying state of the spray dried product and the outlet temperature. Preferably, the inlet temperature is 80-85° C., the outlet temperature is 60-65° C., and the inflow rate of the mixed solution is 720 Ml/hr.

Alternatively, the mixed solution may be granulated using a fluid bed granulator.

Predetermined amounts of microcrystalline cellulose and hard anhydrous silicic acid are fully mixed in the fluid bed granulator while preheating at an inlet temperature of 60-100° C. and an outlet temperature of 40-80° C., and then, the well stirred mixed solution containing the oltipraz and the polymer is allowed to flow in the fluid bed granulator at an inflow rate of 300-1500 Ml/hr. While drying, the oltipraz and the polymer are adsorbed on the microcrystalline cellulose and the hard anhydrous silicic acid to obtain the granules. Preferably, the inlet temperature is 80-85° C., the outlet temperature is 60-65° C., and the inflow rate of the mixed solution is 720 Ml/hr.

Polysorbate or its derivative, or sodium lauryl sulfate may be further added to the mixed solution to increase the solubility of the oltipraz. The amount of polysorbate or its derivative, or sodium lauryl sulfate may be not more than 2.5% by weight in the granules.

A mixing ratio of a mixture of the oltipraz and the polymer to the microcrystalline cellulose may be 1:1-1:3. A mixing ratio of the mixture of the oltipraz and the polymer to the hard anhydrous silicic acid may be 1:0.1-1:1. Preferably, a mixing ratio of the mixture, the microcrystalline cellulose, and the hard anhydrous silicic acid is 1:2:0.5.

The spray dried product or granules produced using the above method may be used in itself or formulated into a tablet or a capsule.

Hereinafter, the present invention will be described in more detail with reference to the following examples. However, these examples are given for the purpose of illustration and are not intended to limit the scope of the invention.

### EXAMPLE 1

### Preparation of a Spray Dried Product (1)

Thirty gram of oltipraz was dissolved in 1.8 L of methylene chloride and 70 g of polyvinylpyrrolidone (molecular weight: 40,000) was dissolved in 200 ml of ethanol, and then, the two solutions were mixed to obtain a mixed solution. Then, the mixed solution was sprayed using a spray dryer (Buch B250, Switzerland) at an inlet temperature of 80° C., an outlet temperature of 60° C., and an inflow rate of 720 Ml/hr to obtain about 30 g of the spray dried product, which has a weight ratio of oltipraz and polyvinylpyrrolidone of 3:7.

### EXAMPLE 2

### Preparation of a Spray Dried Product (2)

A spray dried product having a weight ratio of oltipraz and polyvinylpyrrolidone of 3:7 was prepared in the same manner as in Example 1, except that a polyvinylpyrrolidone-vinyl acetate copolymer was used in place of polyvinylpyrrolidone.

### EXAMPLE 3

### Preparation of a Spray Dried Product (3)

Thirty gram of oltipraz was dissolved in 1.8 L of methylene chloride and 70 g of hydroxypropylmethylcellulose was dissolved in 200 ml of acetone, and then, the two solutions were mixed for 30 minutes to obtain a mixed solution. Then, the mixed solution was sprayed using a spray dryer at an inlet temperature of 80° C., an outlet temperature of 60° C., and an inflow rate of 720 Ml/hr to obtain the spray dried product, which has a weight ratio of oltipraz and hydroxypropylmethylcellulose of 3:7.

### EXAMPLE 4

### Preparation of a Spray Dried Product (4)

Thirty gram of oltipraz was dissolved in 1.8 L of methylene chloride and 30 g of hydroxypropyl-β-cyclodextrin was dissolved in 500 ml of ethanol, and then, the two solutions were mixed for 30 minutes to obtain a mixed solution. Then, the mixed solution was sprayed using a spray dryer at an inlet temperature of 80° C., an outlet temperature of 60° C., and an inflow rate of 720 Ml/hr to obtain the spray dried product, which has a weight ratio of oltipraz and hydroxypropyl-β-cyclodextrin of 1:1.

### EXAMPLE 5

### Preparation of a Spray Dried Product (5)

Ten gram of oltipraz was dissolved in 1.8 L of methylene chloride and, in a separate container, 30 g of polyvinylpyrrolidone (molecular weight: 40,000) and 60 g of γ-cyclodextrin was dissolved in 1 L of 50% ethanol, and then, the two solutions were mixed for 30 minutes in another container to obtain a mixed solution. Then, the mixed solution was sprayed using a spray dryer at an inlet temperature of 80° C., an outlet temperature of 60° C., and an inflow rate of 720 Ml/hr to obtain the spray dried product, which has a weight ratio of oltipraz, polyvinylpyrrolidone (molecular weight: 40,000), and γ-cyclodextrin of 1:3:6.

### EXAMPLE 6

### Preparation of Granules (1)

Thirty gram of oltipraz was dissolved in 1.8 L of methylene chloride and 70 g of polyvinylpyrrolidone (molecular weight: 40,000) was dissolved in 200 ml of ethanol, and then, the two solutions were mixed to obtain a mixed solution. Two hundred gram of microcrystalline cellulose (Avicel PH101) and 50 g of hard anhydrous silicic acid were fully mixed in a fluid bed granulator (FREUND Spir-A-Flow, Japan) while preheating at an inlet temperature of 60° C. and an outlet temperature of 40° C. Next, the mixed solution was sprayed to adsorb the oltipraz dissolved in the polyvinylpyrrolidone, on the microcrystalline cellulose (Avicel PH101) and the hard anhydrous silicic acid. Thus, the dried granules from which the solvent was completely removed were obtained.

### EXAMPLE 7

### Preparation of Granules (2)

Thirty gram of oltipraz was dissolved in 1.8 L of methylene chloride and 70 g of a polyvinylpyrrolidone-vinyl acetate copolymer was dissolved in 200 ml of ethanol, and then, the two solutions were mixed to obtain a mixed solution. Two hundred gram of microcrystalline cellulose (Avicel PH101) and 50 g of hard anhydrous silicic acid were fully mixed in a fluid bed granulator, while preheating at an inlet temperature of 60-100° C. and an outlet temperature of 40-80° C. Next, the mixed solution was sprayed to adsorb the oltipraz dissolved in the polyvinylpyrrolidone-vinyl acetate copolymer, on the microcrystalline cellulose (Avicel PH101) and the hard anhydrous silicic acid. Thus, the dried granules from which the solvent was completely removed were obtained.

### EXAMPLE 8

### Preparation of a Spray Dried Product (6)

Forty-five gram of oltipraz was dissolved in 2.7 L of methylene chloride and 45 g of polyvinylpyrrolidone (molecular weight: 40,000) and 60 g of citric acid were added to 300 ml of ethanol and dissolved while milling using a high-speed emulsifier. Then, the two solutions were mixed for 30 minutes to obtain a mixed solution. Then, the mixed solution was sprayed using a spray dryer at an inlet temperature of 80° C., an outlet temperature of 60° C., and an inflow rate of 720 Ml/hr to obtain the spray dried product, which has a weight ratio of oltipraz, polyvinylpyrrolidone and citric acid of 45:45:60.

### EXAMPLE 9

### Preparation of a Spray Dried Product (7)

Forty-five gram of oltipraz was dissolved in 2.7 L of methylene chloride and 45 g of polyvinylpyrrolidone-vinyl acetate copolymer and 10 g of citric acid were added to 300 ml of ethanol and dissolved while milling using a high-speed emulsifier. Then, the two solutions were mixed for 30 minutes to obtain a mixed solution. Then, the mixed solution was sprayed using a spray dryer at an inlet temperature of 80° C., an outlet temperature of 60° C., and an inflow rate of 720 Ml/hr to obtain the spray dried product, which has a weight ratio of oltipraz, polyvinylpyrrolidone-vinyl acetate copolymer and citric acid of 45:45:10.

### EXAMPLE 10

### Preparation of Tablets (1)

Forty-eight point five parts by weight of microcrystalline cellulose for direct compression, 6.0 parts by weight of sodium gluconate, and 1.61 parts by weight of magnesium stearate were mixed with 100 parts by weight of the spray dried product containing oltipraz, prepared in of Example 1. The resultant mixture was compressed to form tablets having a hardness of 10 Kp.

### EXAMPLE 11

### Preparation of Tablets (2)

Forty-eight point five parts by weight of microcrystalline cellulose for direct compression, 6.0 parts by weight of sodium gluconate, and 1.61 parts by weight of magnesium stearate were mixed with 100 parts by weight of the spray dried product containing oltipraz, prepared in of Example 2. The resultant mixture was compressed to form tablets having a hardness of 10 Kp.

### EXAMPLE 12

### Preparation of Capsules (1)

Thirty gram of microcrystalline cellulose and 3 g of magnesium stearate were mixed with 100 g of the spray dried product containing oltipraz, prepared in of Example 1. The resultant mixture was filled in capsules to obtain oltipraz capsules.

### EXAMPLE 13

### Preparation of Capsules (2)

Five gram of magnesium stearate was mixed with 350 g of the oltipraz granules prepared in of Example 6. The resultant mixture was filled in capsules to obtain oltipraz capsules.

### COMPARATIVE EXAMPLE 1

Thirty gram of oltipraz was pulverized to particles with an average particle size of 5 μm using an air jet impact mill (SANKI Jet-miller, Japan) and suspended in physiological saline solution.

### EXPERIMENTAL EXAMPLE 1

### Evaluation of Solubility and Bioavailability

To evaluate the solubility of each of the tablets prepared in Examples 10 and 11, a dissolution test was carried out. Also, to evaluate the bioavailability of each of the spray dried products prepared in Examples 1 and 2, an animal test was carried out.

A. Dissolution test

The dissolution test was carried out according to the Dissolution test (Second method) among the General tests described in The Korean Pharmacopoeia. 900 ml of 3% sodium lauryl sulfate was used as a dissolution solution for a tablet prepared using the oltipraz prepared in Comparative Example 1, the tablet prepared in Example 10, and the tablet prepared in Example 11 and the dissolution was performed at 100 revolutions/min for 120 minutes.

The dissolution solution was taken at 0, 15, 30, 60, 90, 120 minutes after the start of the dissolution test and filtered, and then, each of the resultant filtrates was analyzed using high performance liquid chromatography (HPLC).

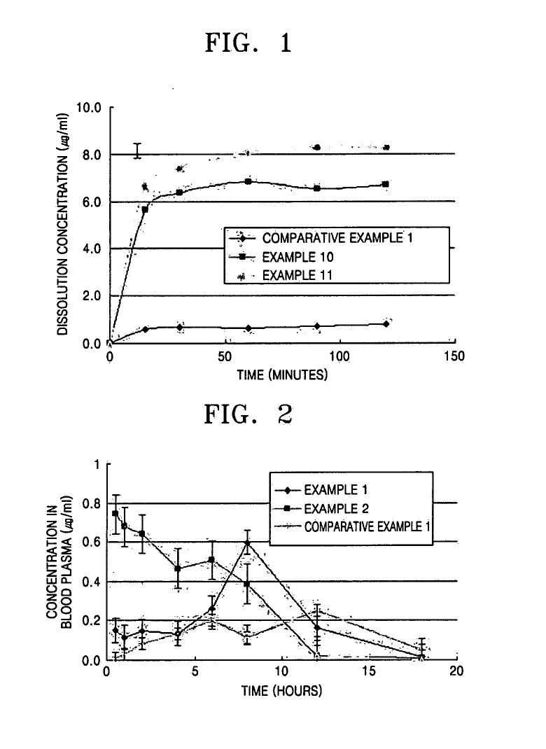

FIG. 1 is a graph of time vs. dissolution concentrations for the tablets prepared in Comparative Example 1, Example 10, and Example 11. It was confirmed from FIG. 1 that the tablets prepared in Examples 10 and 11 had a remarkably higher dissolution rate and a dissolution amount than the tablet prepared using the oltipraz prepared in Comparative Example 1.

B. Animal test

Each of the spray dried product prepared in Example 1, the spray dried product prepared in Example 2, and the product prepared in Comparative Example 1 was orally administered to starved rats weighing 180-230 g at an dose of 50 equ.mg/5 ml/kg. Then, a concentration of oltipraz in blood plasma was measured for 30 hours.

The results are shown in Table 2 and FIG. 2.

TABLE 2

| | Oltipraz composition | | |
|---|---|---|---|
| | (saline solution, N = 3) | | |
| | | | Comparative |
| | Example 1 | Example 2 | Example 1 |
| Cₘₐₓ (μg/ml) | 0.45 | 0.52 | 0.3 |
| Tₘₐₓ (hr) | 1.8 | 1 | 6 |
| T_{2/1} (hr) | 3.65 | 4.4 | 3.9 |
| AUC_{0-∞} (ug · hr/ml) | 5.2 | 5.2 | 3.5 |

The spray dried products prepared in Examples 1 and 2 had higher Cₘₐₓ and shorter Tₘₐₓ than the micronized powder prepared in Comparative Example 1. Further, they have AUC at least 1.5 times higher than the micornized powder prepared in Comparative Example 1.

It was confirmed from the above results that the oltipraz tablets prepared using a preparation method according to embodiments of the present invention had a remarkably higher bioavailability than the oltipraz prepared in Comparative Example 1.

### EXPERIMENTAL EXAMPLE 2

### Scanning Electron Microscope (SEM) Photography

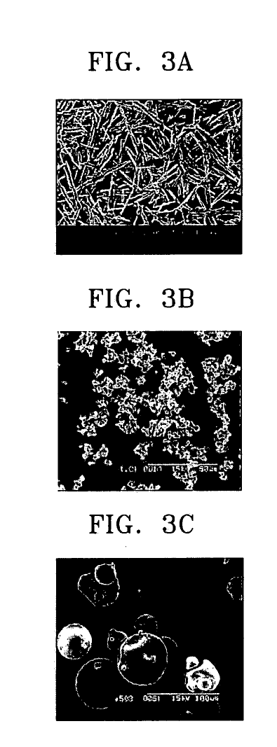

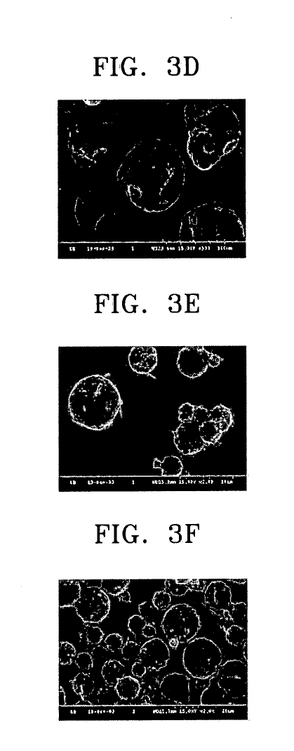

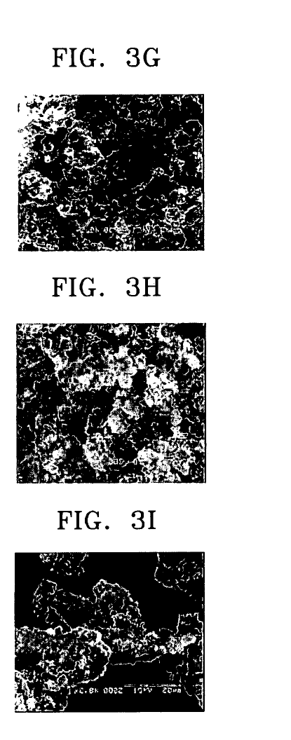

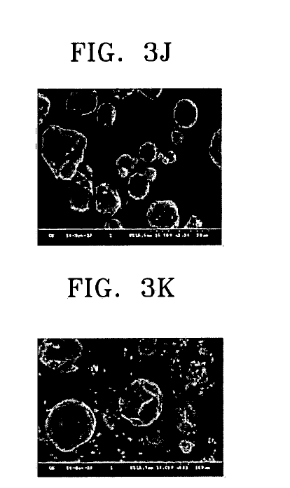

SEM photos were taken for oltipraz powders used as a raw material in the above Examples (FIG. 3A), the oltipraz pulverized using an air jet impact mill (at a rate of recovery of 2.5 kg/hr and a nozzle pressure of 0.70 mPa) (FIG. 3B), polyvinylpyrrolidone (molecular weight: 40,000) (FIG. 3C), a polyvinylpyrrolidone-vinyl acetate copolymer (FIG. 3D), the spray dried product prepared in Example 1 (FIG. 3E), the spray dried product prepared in Example 2 (FIG. 3F), the spray dried product prepared in Example 5 (FIG. 3G), the spray dried product prepared in Example 3 (FIG. 3H), the granules prepared in Example 6 (FIG. 3I), the spray dried product prepared in Example 9 (FIG. 3J), and a mixture of oltipraz and polyvinylpirrolidone-vinyl acetate copolymer in a ratio of 3:7 (FIG. 3K).

By forming a spray dried product using oltipraz, which has a structure of needle-like crystal, and polyvinylpyrrolidone or polyvinylpyrrolidone-vinyl acetate copolymer, which has a circular shape and is amorphous, the needle-like oltipraz was not observed, which demonstrates that the oltipraz is in an amorphous form.

The spray dried product has a clear difference in the structure of oltipraz from the simple mixture of oltipraz and polyvinylpirrolidone-vinyl acetate copolymer in a ratio of 3:7 illustrated in FIG. 3K.

EXPERIMENTAL EXAMPLE 3

### Measurement of Crystallinity Using an X-Ray Diffractor

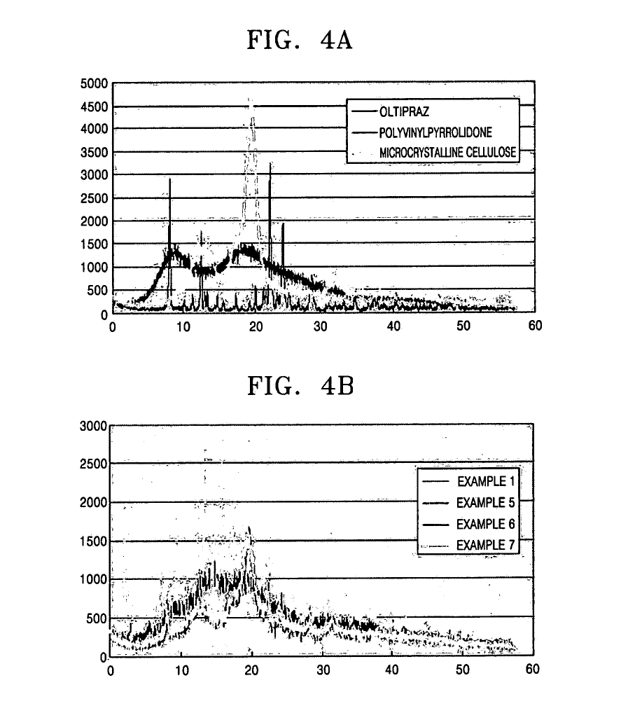

To confirm a reduction in crystallinity, oltipraz used as a raw material in the above Examples, polyvinylpyrrolidone (molecular weight: 40,000), and microcrystalline cellulose (FIG. 4A) and the spray dried product prepared in Example 1, the spray dried product prepared in Example 5, the granules prepared in Example 6, and the granules prepared in Example 7 (FIG. 4B) were measured for their crystallinity using an X-ray diffractor (Rigaku D/MAX-IIIB). The results are shown in FIGS. 4A and 4B.

Crystallinity of each of the compositional components used in Examples 1-13 can be confirmed from FIG. 4A. Oltipraz exhibited sharp peaks, which demonstrate that the oltipraz has high crystallinity. Polyvinylpyrrolidone, which was used in the spray dried product of Example 1, exhibited broad peaks, which demonstrate that it is amorphous.

Referring to FIG. 4B, the spray dried product prepared using polyvinylpyrrolidone (molecular weight: 40,000) in Example 1 is little crystallized and the spray dried product prepared in Example 5 is crystallized due to the γ-cyclodextrin. The granules prepared in Examples 6 and 7 are slightly crystallized due to the effect of microcrystalline cellulose.

As described above, according to the present invention, low-crystallinity oltipraz or amorphous oltipraz can be prepared, thereby increasing the solubility and bioavailability of oltipraz, which has a low solubility.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of preparing low-crystallinity oltipraz or amorphous oltipraz, comprising:
obtaining a mixed solution containing oltipraz and a water-soluble polymer or a water-insoluble polymer in a solvent, the solvent being an organic solvent or purified water; and
solid-dispersing the oltipraz in the polymer.

2. The method of claim 1, wherein in the solid-dispersing, the mixed solution is spray dried using a spray dryer or is granulated using a fluid bed granulator.

3. The method of claim 1, wherein the mixed solution further comprises an absorption enhancer.

4. The method of claim 3, wherein the absorption enhancer includes at least one compound selected from the group consisting of ascorbic acid, citric acid, xylitol, and polyethylene glycol or its derivative.

5. The method of claim 1, wherein the water-soluble polymer includes at least one polymer selected from the group consisting of polyvinylpyrrolidone or its derivative, a polyvinylpyrrolidone-vinyl acetate copolymer, alginic acid, alginate or its derivative, α-cyclodextrin or its derivative, β-cyclodextrin or its derivative, γ-cyclodextrin or its derivative, polyoxyethylene-polyoxypropylene copolymer, polyethylene glycol or its derivative, polyvinyl alcohol, xanthan gum, arabic gum, or a combination thereof.

6. The method of claim 5, wherein the polyvinylpyrrolidone has a molecular weight of 2,500-3,000,000.

7. The method of claim 5, wherein the polyvinylpyrrolidone-vinyl acetate copolymer has a molecular weight of 30,000-50,000.

8. The method of claim 5, wherein the alginate derivative is an ethylene or propylene derivative of sodium alginate and has a molecular weight of 20,000-200,000.

9. The method of claim 5, wherein the β-cyclodextrin derivative is a propylene derivative of β-cyclodextrin or a metylated derivative of β-cyclodextrin.

10. The method of claim 5, wherein the polyoxyethylene-polyoxypropylene copolymer has an oxyethylene content of 45-75%.

11. The method of claim 5, wherein the polyethylene glycol or its derivative has a molecular weight of 200-90,000.

12. The method of claim 11, wherein the polyethylene glycol derivative is an esterificated derivative of polyethylene glycol.

13. The method of claim 1, wherein the water-insoluble polymer includes at least one one selected from the group consisting of cellulose or its derivative, polymethacrylate, and polyalkylacrylate.

14. The method of claim 13, wherein the cellulose derivative is cellulose acetate, cellulose acetate phthalate, hydroxypropylene methylcellulose, hydroxypropylene methylcellulose phthalate, ethylcellulose, methylcellulose, or hydroxypropylene cellulose.

15. The method of claim 13, wherein the cellulose derivative is hydroxypropylene methylcellulose having a viscosity of 5-50 cps.

16. The method of claim 1, wherein the concentration of the water-soluble polymer or water-insoluble polymer in the mixed solution is 10-90 parts by weight based on 100 parts by weight of oltipraz.

17. The method of claim 3, wherein the concentration of the water-soluble polymer or water-insoluble polymer in the mixed solution is 5-90 parts by weight and the concentration of the absorption enhancer in the mixed solution is 5-90 parts by weight, respectively, based on 100 parts by weight of oltipraz.

18. A method of using the low-crystallinity oltipraz or amorphous oltipraz claim 1 in preparing a tablet or a capsule.
